(19) United States
(12) Patent Application Publication
(10) Pub. No.: US 2005/0084983 A1
(43) Pub. Date: Apr. 21, 2005

(54) Urine test for the diagnosis of prion diseases

(76) Inventors: Ruth Gabizon, Jerusalem (IL); Gideon M Shaked, Moshav Ora (IL)

Correspondence Address: BANNER & WITCOFF, 1001 G STREET N W, SUITE 1100, WASHINGTON, DC, 20001, US

(21) Appl. No.: 10/399,321

(22) PCT Filed: Oct. 21, 2001
(86) PCT No.: PCT/IL01/00968

(30) Foreign Application Priority Data
Oct. 22, 2000 (IL) 139185
Mar. 12, 2001 (IL) 141950

(51) Int. Cl. G01N 33/53; G01N 33/543

(52) U.S. Cl. 436/518

(57) ABSTRACT

The present invention relates to a method for detecting the presence of the abnormal isoform of prion protein (PrPSC) in a urine sample of a subject. The method of the invention comprising the steps of: (a) providing a urine sample of said subject; (b) isolating from said sample all proteins, preferably, isolating proteins having a molecular weight higher than about 8 Kda; (c) optionally, and preferably, subjecting the proteins obtained in step (b) to protease digestion, and isolating from the mixture obtained in step (c) any protease resistant proteins; and (d) detecting the presence of PrPSC in the protease resistant fraction obtained in step (c) by a suitable detection technique. Furthermore, the invention further relates to methods for diagnosing a prion disease in a subject and for screening donors of blood samples for the presence of prion diseases. The invention further provides for a diagnostic kit for diagnosing a prion disease in a subject.

### FIELD OF THE INVENTION

The present invention is concerned with a method for the diagnosis of prion diseases by detecting the protease-resistant core of PrP^{SC} and/or some metabolites thereof in urine samples.

### BACKGROUND OF THE INVENTION

Prion diseases, also known as TSEs (transmissible spongiform encephalopathies), are a group of fatal neurodegenerative diseases of animals and humans. Among the animal diseases, the most prevalent today is BSE (bovine spongiform encephalopathy) also known as the “Mad Cow Disease”. Although less than 100 patients have been diagnosed to date to be BSE-infected, the number of individuals incubating the disease may be millions. Another animal prior disease is scrapie in sheep, which after transmission to rodents constitutes the main experimental prior animal model.

In humans, the most prevalent prion disease is CJD (Creutzfeldt Jakob Discase), which can be manifested either sporadically (about 1 patent per year); genetically (via mutations in the prion protein PrP gene); or in transmissible form, as in the BSE affected cases. It is a well known experimental fact that the incubation of prior diseases in humans and large animals can last for decades.

Prion diseases are believed to be caused by the accumulation in the brain of PrP^{SC}, an abnormally folded isoform of PrP^{C}, a GPI anchored protein of unknown function. It has been postulated that prion diseases propagate by the conversion of PrP^{C} molecules into protease-resistant and insoluble PrP^{SC} by an as yet unknown mechanism. The proteinase K (PK) resistant PrP in prion diseases was described by McKinley et al. [Cell 35(1):57-62 (1988)]. Immunoblotting of a Proteinase K-digested brain sample infected with a prion disease with an anti-PrP antibody, reveals a characteristic N-terminally truncated PrP protein (the protease resistant core of PrP^{SC}, denominated PrP 27-30), which is not present in controls or in individuals affected with any other neurological disease.

To date, the diagnosis of prion diseases was based on the presence of this characteristic protease-resistant PrP in brain biopsies, as well as on clinical criteria. Current methods for the conclusive identification of Prion diseases include mostly a post-mortem analysis of the patient's brain homogenate. Clinical symptoms of the disease can many times be misleading. Evidently, sampling brain tissue from the living patient involves a painful and risky surgical procedure and, moreover, does not give a definite answer since the distribution of PrP^{SC} in the brain is not homogenous. All commercial tests used to date are based on brain presence of protease resistant PrP, for example the Prion-Test of Prionics AG, Switzerland (which company is in charge of Most European active surveillance for BSE cases), which is an immunological test for the detection of prions in brain and spinal cord tissue, and is mainly used for BSE and scrapie diagnostics. Since the incubation period in prion diseases is very long (years), it is possible that there is a large number of asymptomatic human and animal carriers. There exists therefore a need for developing a simple and readily available pre-clinical and clinical diagnostic test for the disease. The need for such an in-vivo test has been reinforced since the reports of the first cases of variant Creutzfeldt Jakob disease (vCJD) in 1996 [Zeidler, M., et al., Lancet 350(9082), 908-10 (1997); Bruce, M. E., et al., Nature 389(6650), 498-501 (1997); Ironside, J. W., et al., Histopathology 37(1), 1-9 (2000)]. vCJD is a fatal neurodegenerative disease believed to be caused by the consumption of BSE contaminated meat, and the incubation time between infection to clinical symptoms may be as long as decades [Bruce, M. E., et al., Nature ibid (1997)]. As opposed to cattle, the incubating individuals will be present for many years, donating blood and in some cases other organs to the non-affected population. Additionally, such test is important for the food industry, and would enable detecting BSE in bovine animals such as cows and sheep, and to prevent marketing of infected meat and diary products of these animals.

Therefore, a major object of the present invention is the development of a reliable, non-invasive method for diagnosing prion diseases which will allow the pre-clinical and clinical diagnosis of the disease in humans and in animals.

Since most urine proteins originate from blood, the present inventors speculated that some PrP^{SC}, either from brain or from a peripheral organ, is released during the incubation period into the blood serum in a non-aggregated form, although at low and undetectable concentrations. Due to its protease resistance, PrP^{SC} is not digested by blood proteases. However, since the MW of PrP is below the cutoff size for filtering through kidney cells (about 40 kDA) [Berne, R. M., and Levy, M. N. Physiology, 4th Ed (1998)], PrP may subsequently be secreted into the urine and thereby be concentrated, as other proteins, at about 120 folds of its concentration in blood [Kocisko. D. A., et al., Nature 370(6489), 471-4 (1994)]. The concentration by the kidney makes possible to detect PrP^{SC} in urine more easily than in blood.

Thus, as will become apparent as the description proceeds, the present inventors have identified a prion specific protease resistant PrP isoform in the urine of prion infected animals and humans (UPrP^{SC}), which may be used for the in-vivo early diagnosis of ill as well as seemingly healthy but prion infected individuals. Moreover, the present invention shows that this protease resistant isoform UPrP^{SC}, can be detected, following a specific enrichment procedure, in the urine of scrapie-infected hamsters, BSE-infected cattle and humans suffering from CJD. This specific enrichment procedure, according to the present invention may include dialysis of the sample through membrane having a pore range of about 6 KDa to about 8 KDa. The present invention further shows that UPrP^{SC} was also found in urine of hamsters inoculated with prions long before the appearance of clinical signs. These findings strongly indicate the possibility of using the method of the invention also for pre-clinical diagnosis.

The theoretical possibility for diagnosis of prion diseases in variety of body fluids, such as urine, has been mentioned in several patent documents. EP 0854364, for example, discloses a diagnostic method for neuro-degenerative disorders such as Alzheimer's disease and prion diseases. This method is based on concentrating a protein associated with the specific neuro-degenerative disease (such as PrP in prion diseases and APP in Alzheimer's disease), in a sample (urine, for example). The concentration is carried out by contacting the sample with a solid, non-buoyant particulate material having free ionic valencies such as calcium phosphate. However, this patent exemplifies the detection of only the Alzheimer's disease associated peptide APP. WO 93/23432 discloses a diagnostic method for prion diseases in different body fluids such as CSF (cerebrospinal fluid) and theoretically, urine. Similarly to EP 0854364, this method is based on concentrating the prion protein by ammonium sulfate precipitation and affinity chromatography. This publication exemplifies CSF as a sample.

However, contrary to the prior art methods, the present invention clearly demonstrates the detection of the aberrant protease resistant urine isoform UPrP^{SC} in urine samples of prion infected animals and humans. Furthermore, as shown by the present invention, dialysis of the urine seems to improve the detection procedure. Therefore, the present inventors propose that UPrP^{SC} is present in a semi-denatured form, probably due to the relative high concentrations of urine denaturing agents, and is subsequently re-natured for example by the dialysis step. Thus, the specific enrichment of the urine sample according to the present invention provides a novel and reliable method for the detection of different prion diseases by a non-invasive procedure.

### SUMMARY OF THE INVENTION

The invention relates to a method for detecting the presence of the abnormal isoform of prion protein (PrP^{SC}) in a urine sample of a subject, said method comprising the steps of: (a) providing a urine sample of said subject; (b) isolating from said sample proteins; and (c) detecting the presence of PrP^{SC} in the protein mixture obtained in step (b) by a suitable detection technique.

A preferred embodiment relates to the method of the present invention, further comprising the step of subjecting the proteins obtained in step (b) to protease digestion.

In a specifically preferred embodiment, the invention relates to a method for detecting the presence of the abnormal isoform of prion protein (PrP^{SC}) in a urine sample of a subject, said method comprising the steps of (a) providing a urine sample of said subject; (b) isolating from said sample all proteins having a molecular weight higher than about 8 KDa; (c) subjecting the proteins obtained in step (b) to protease digestion; (d) isolating from the mixture obtained in step (c) any protease resistant proteins; and (e) detecting the presence of PrP^{SC} in the protease resistant fraction obtained in step (d) by a suitable detection technique.

In step (b) of the method of the invention the proteins are preferably isolated by subjecting the urine sample to dialysis and precipitating the proteins from the dialysate. Optionally, prior to the protein precipitation, a carrier may be added to the dialysate for stabilizing the PrP^{SC}. The dialysis is preferably performed using a membrane having a pore range of from about 6 KDa to about 8 KDa.

The proteins may be precipitated from the dialysate by ultracentrifuging the same, for example for about 1 hour at 100,000×g at 4° C. Alternatively the proteins may be precipitated by any suitable protein precipitation technique. As a preferred embodiment proteins according to the invention may be precipitated by any one of methanol, TCA (Trichloracetic acid) or by any other precipitation method. Preferably, proteins may be precipitated by methanol, for example by the addition of methanol and freezing the sample to about −80° C. for about 1 hour, and subsequently centrifuging at 3000×rpm for about 30 minutes.

The protein digestion is preferably performed by treating the sample with proteinase K, for example by adding proteinase K in concentration of up to 40 μg/ml and continuing digestion for about 30 min at 37° C.

The presence of the PrP^{SC} protease-resistant core in said non-digested fraction is preferably detected by immunoassay, for example by immunoblot SDS PAGE analysis, using monoclonal antibodies that specifically bind to the protease-resistant core of PrP^{SC}, for example 3F4 or GH4 monoclonal antibodies.

The invention also relates to a method for diagnosing a prion disease in a subject comprising the steps of (a) obtaining a urine sample of said subject; and (b) detecting the presence of the abnormal isoform of prion protein (PrP^{SC}) in said urine sample by the method of the invention, whereby the presence the PrP^{SC} protein in said sample indicates that said subject carries a prion disease. In a preferred embodiment, said prion disease may be any TSE disease. The subject may be a human subject, for example a CJD, vCJD, GSS or FFI carrier or an individual infected with BSE. Alternatively the subject may be an animal infected with BSE, scrapie or any other TSE disease. The method of the invention further enables detection of different prion disease prior to or after onset of clinical symptoms.

In yet a further embodiment that invention relates to a method for screening donors of blood samples for the presence of a prion disease in said donor comprising the steps of: (a) obtaining a urine sample from said donor; (b) detecting the presence of the abnormal isoform of prion protein (PrP^{SC}) in said urine sample by the method of the invention; and matching the results of the detection performed in step (b) to said blood sample.

Still further, the invention relates to a method for detecting the presence of metabolites of the abnormal isoform which is probably a pathogenic isoform of prion protein (PrP^{SC}) in a urine sample of a subject, said metabolites being unique for human prion disease carriers. In a preferred embodiment such human prion disease may be CJD or vCJD. This method comprises the steps of: (a) providing a urine sample of said subject; (b) isolating from said sample all proteins having a molecular weight higher than about 8 KDa; and (c) detecting the presence of said metabolites of PrP^{SC} in the protein sample obtained in step (b) by a suitable detection technique.

In this embodiment, in step (b) said proteins may be isolated by subjecting the urine sample to dialysis and precipitating the proteins from the dialysate, for example by ultracentrifuging the dialysate, specifically for about 1 hour at 100,000×g at 4° C., or by any other suitable precipitation method. Preferred protein precipitation method may be methods such as methanol or TCA (Trichloracetic acid) precipitation. A specifically preferred technique for precipitation is methanol precipitation, specifically by the addition of methanol to the sample, freezing to about −80° C. for about 1 hour, and subsequently centrifuging at 3000×rpm (rounds per minute) for about 30 minutes.

The detection of the presence of the said metabolites of PrP^{SC} protease-resistant core in said protein sample is preferably by immunoassay, particularly SDS PAGE, using monoclonal antibodies that specifically bind to the specific metabolites of PrP^{SC} found in urine of prion disease carriers, for example 6H4 monoclonal antibodies.

The invention further relates to a method for diagnosing a prion disease in a subject comprising the steps of: (a) obtaining a urine sample of said subject; and (b) detecting the presence of metabolites of the abnormal isoform of prion protein (PrP^{SC}) that are unique for piron disease patients in said urine sample by a method of the invention; whereby the presence of said PrP^{SC} protein metabolites in said sample indicates that said subject carries prion disease.

According to a preferred embodiment, the method of the invention is intended for detection of the presence of metabolites unique for CJD and vCJD.

Still further, the invention relates to a method for screening donors of blood samples for the presence of prion disease in said donor. This method comprises the steps of: (a) obtaining a urine sample from said donor; (b) detecting the presence of metabolites of the abnormal isoform of prion protein (PrP^{SC}) that are unique for prion disease patients in said urine sample by a method of the invention; and matching the results of the detection performed in step (b) to said blood sample.

In another embodiment the invention relates to a diagnostic kit for detecting the presence of the abnormal isoform of prion protein (PrR^{SC}) in a urine sample of a subject, said kit comprising means for isolating from said urine sample all proteins; optionally, a carrier for stabilizing the PrP^{SC}; means for detecting the presence of PrP^{SC} in the non-digested fraction; and instructions for carrying out the detection of the presence of PrP^{SC} in the urine samples.

In another preferred embodiment the invention relates to a diagnostic kit for detecting the presence of the abnormal isoform of prion protein (PrP^{SC}) in a urine sample of a subject, said kit comprising means for isolating from said urine sample all proteins having a molecular weight higher than about 8 KDa; optionally, a carrier for stabilizing the PrP^{SC} in the dialysate; means for detecting the presence of PrP^{SC}; and instructions for carrying out the detection of the presence of PrP^{SC} in the urine samples.

In yet another specifically preferred embodiment the invention relates to a diagnostic kit for detecting the presence of the abnormal isoform of prion protein (PrP^{SC}) in a urine sample of a subject, said kit comprising means for isolating form said urine sample all proteins having a molecular weight higher than about 8 KDa; optionally, a carrier for stabilizing the PrP^{SC} in the dialysate; a protease for digesting the protein isolate; means for isolating from the protein digest any protease digest any protease resistant proteins; means for detecting the presence of PrP^{SC} in the protease resistant fraction; and instructions for carrying out the detection of the presence of PrP^{SC} in the urine samples.

In the kit of the invention, protease is preferably proteinase K and said means for detecting the presence of PrP^{SC} comprise reagents for detecting PrP^{SC} by immunoassay, such as antibodies that specifically react with the protease-resistant core of PrP^{SC}.

In another embodiment the invention relates to a diagnostic kit for detecting the presence of metabolites of the abnormal isoform of prion protein )PrP^{SC}) that are unique for human prion disease carriers, in a urine sample of a subject, said kit comprising: means for isolating from said urine sample all proteins having a molecular weight higher than about 8 KDa and means for detecting the presence of PrP^{SC} metabolites that are unique for human prion disease carriers, preferably CJD and vCJD, in the obtained protein sample. The means for detecting the presence of said PrP^{SC} metabolites preferably comprise reagents for detecting said PrP^{SC} metabolites by immunoassay, for example antibodies that specifically react with the metabolites of PrP^{SC} that are unique for human prion disease carriers.

According to a preferred embodiment, said human prion disease may be CJD or vCJD.

### BRIEF DESCRIPTION OF THE DRAWINGS

The present invention will be described in more detail on hand of the attached drawings in which:

FIG. 1 shows an immunoblot analysis of PrP^{SC} in urine samples from Scrapie-infected hamsters (Sc) or from normal controls (N) using 3F4 monoclonal antibodies. Samples were either treated (+) or not treated (−) with proteinase K (PK).

FIG. 2 shows an immunoblot analysis of PrP^{SC} in urine samples from homozygous (HOZ) or heterozygous (HTZ) human patients suffering from CJD, MS (multiple sclerosis), stroke (str) and healthy individuals (Norm) using 3F4 monoclonal antibodies. Hamster brain extracts (Ham br) was used as positive control. Samples were either treated (+) or not treated (−) with proteinase K (PK).

FIG. 3 shows an immunoblot analysis of PrP^{SC} in urine samples from homozygous (HOZ) CJD patients and healthy individuals (Norm) using 3F4 monoclonal antibodies. Hamster brain extracts (Ham br) were used as positive control. Samples were either treated (+) or not treated (−) with proteinase K (PK). Blue pre-stained marker (Novex) was used as molecular weight marker (M).

FIG. 4A-C shows protease resistant PrP in urine or brain samples of TSE affected humans and animals.

FIG. 4A: shows freshly frozen urine samples from hamsters, humans, and cattle that were enriched for protease resistant PrP as described in the experimental procedures. All samples were digested in the presence or absence of Proteinase K (+ or − PK, respectively), and immunoblotted with either anti-PrP mAb 3F4 (hamster and human samples) or 6H4 (bovine samples). (1) Homozygous E200K CJD patient; (2) Heterozygous E200K CJD patient; (3) Human control; (4) Scrapie sick hamster; (5) Normal hamster; (6) BSE sick cattle; (7) Normal cattle.

FIG. 4B: shows similar analysis performed using 5 μl of a 10% brain samples (1) Homozygous CJD patient; (2) Heterozygous CJD patient; (3) Human control; (4) Scrapie sick hamster; (5) Normal hamster; (6) Kidney sample from scrapie sick hamster;

FIG. 4C: shows blocking experiment performed in human brain sample (b) and human urine sample (u). Samples were immunoblotted with mAb 3F4 in the absence (1) or the presence (2) of 10 μg/ml of the peptide comprising the 3F4 epitope. Molecular Weight markers (top to bottom); 36 kDa, 30 kDa.

FIG. 5 shows scrapie hamster urine samples that were enriched for UPrP^{SC} with and without dialysis step (Dia). Samples were digested in the presence (+) or absence (−) of proteinase K (PK) as described in methods.

FIG. 6 shows an immunoblot analysis of PrP^{SC} obtained from CJD patients that were either treated or not treated with DMSO for 1 day (+DMSO (1 d)) and healthy individuals (Norm), using 6H4 antibodies as compared to 3F4 antibodies. Hamster brain extracts (Ham br) were used as positive control. Samples were precipitated using methanol and were either treated (+) or not treated (−) with proteinase K (PK). Blue pre-stained marker (Novex) was used as molecular weight marker (M).

FIG. 7A-B shows that prion specific PrP can be detected during scrapie incubation time (Inc T) in days (d). Urine samples were collected weekly from Syrian hamsters inoculated either i.c. (7A), or i.p. (7B). Samples were immunoblotted with αPrP mAb 3F4. Arrows represent the onset of clinical signs (Clin sig). Molecular weight markers (top to bottom): 36 kDa, 30 kDa.

FIG. 7A; shows Syrian hamsters inoculated i.c. (intra-cerebally) with hamster 263K prions, and enriched for UPrP^{SC}.

FIG. 7B: shows Syrian hamsters inoculated i.p. (intraperitoneally) with hamster 263K prions, and enriched for UPrP^{SC}.

FIG. 8A-B shows i.c. (intra-cerebally) inoculation of Syrian hamsters with UPrP^{SC}.

Syrian hamsters were inoculated with equivalent amounts of PK resistant PrP from brain or urine of scrapie infected hamsters. All samples were immunoblotted with 1:5000 mAb 3F4.

FIG. 8A: shows PK resistant PrP^{SC} equivalents originating from 5μl of 10% hamster brain homogenate (1) as compared to 2 ml scrapie hamster urine (2).

FIG. 8B: shows brain sample from a scrapie infected hamster (1); urine samples collected (at 60 dpi) from hamsters inoculated with UPrP^{Sc} (2); or brain sample of one of the animals inoculated with UPrP^{SC} (3). All samples were digested in the presence (+) or absence (31) of PK. Molecular weight markers (top to bottom); 36 kDa, 30 kDa.

### DETAILED DESCRIPTION OF PREFERRED EMBODIMENTS

The inventors have now surprisingly found, and this is an object of the invention, that PrP^{SC}, the aberrant isoform and the only known marker for prion diseases, can be identified in the urine of hamsters infected with scrapie, as well as in the urine of humans sick with CJD. In addition, some metabolites of the PrP^{SC} could be detected in the urine of CJD patients, while they were absent from urine of normal individuals.

Thus, in a first aspect, the invention relates to a method for detecting the presence of the abnormal isoform of prion protein (PrP^{SC}) in a urine sample of a subject, said method comprising the steps of: (a) providing a urine sample of said subject; (b) isolating or concentrating from said sample proteins; and (c) detecting the presence of PrP^{SC} in the protein mixture obtained in step (b) by a suitable detection technique.

A preferred embodiment relates to the method of the present invention, further comprising the step of subjecting the proteins obtained in step (b) to protease digestion.

In a specifically preferred embodiment, the invention relates to a method for detecting the presence of the abnormal isoform of prion protein (PrP^{SC}) in a urine sample of a subject, said method comprising the steps of (a) providing a urine sample of said subject; (b) isolating or concentrating from said sample all proteins having a molecular weight higher than about 8 KDa; (c) subjecting the proteins obtained in step (b) to protease digestion; (d) isolating from the mixture obtained in step (c) any protease resistant proteins; and (c) detecting the presence of PrP^{SC} in the protease resistant fraction obtained in step (d) by a suitable detection technique.

In one preferred embodiment, the proteins may be isolated from the urine sample by subjecting the sample to dialysis and precipitating the proteins from the dialysate. The dialysis may be preferably performed using a membrane having a pore range of from about 6 KDa to about 8 KDa.

Following the dialysis, the proteins may be precipitated from the dialysate by ultracentrifuging, for example for about 1 hour at 100,000×g at 4° C., or by any other suitable protein precipitation technique. As a non-limitinig example such protein precipitation techniques may be by any one of methanol or TCA (Trichloracetic acid). Methanol precipitation is preferred for example, by adding methanol and freezing the sample to about −80° C. for about 1 hours, and subsequently centrifuging at 3000×rpm for about 30 minutes.

In yet another preferred embodiment proteins are precipitated using TCA. Briefly according to a modified protocol, sample is diluted with 10% TCA, kept for two hours on ice, and subsequently centrifuged at 14000 rpm at 4° C. After discarding the supernatant the pellet is subjected twice to ethanol precipitation [the modified protocol is based upon the TCA precipitation protocol on: Antibodies, a laboratory manual, editors: Ed Harlow, David Lane, Cold spring harbor laboratory (1988)].

Optionally, prior to the protein precipitation, the PrP^{SC} may be stabilized by adding a carrier to the dialysate. For example, such carrier may be brain extract of PrP ablated mice.

After the said proteins are isolated, they are subjected to digestion by a protease, preferably by proteinase K, for example by adding to the sample proteinse K at a concentration of about 40 μg/ml and continuing digestion for about 30 min at 37° C.

Preferably, the presence of the PrP^{SC} protease-resistant core in said non-digested fraction is detected by immunoassay, for example by immunoblot SDS PAGE, employing monoclonal antibodies that specifically bind to the protease-resistant core of PrP^{SC}, preferably the monoclonal antibodies 3F4 or 6H4. The presence of this protein can also be identified by dot blot immunoassays, and by specifically adapted ELISA test.

The invention thus provides an efficient, non-invasive method for the diagnosis of prion diseases. It may be appreciated that while the rationale underlying the method of the present invention is yet unclear, it is possible that the PrP^{SC} is secreted from the brain cells during the pre-clinical or clinical stage of the disease, and since this protein is protease-resistant, it is cleared into the urine before it can be digested in the blood.

In a further embodiment, the method of detection of the present invention may be used for diagnosing a prion disease in a human or animal subject, by obtaining a urine sample of the subject and detecting the presence of the abnormal isoform of prion protein (PrP^{SC}) in said urine sample by the detection method the invention, the presence of the PrP^{SC} protein in the urine of the subject indicating that said subject carries a prion disease. This abnormal isoform is probably a pathogenic isoform of the prion protein. Thus, the invention provides a method for the detection of different prion diseases before or after onset of clinical symptoms.

The prion disease according to a preferred embodiment is a TSE disease. TSE disease may be as a non limiting example any one of CJD, FFI, GSS in human. In a non-human animal TSE disease may be any one of BSE, Scrapie, CWD (Chronic Wasting Disease) of mule, deer and elk and TME (Transmissible Mink Encephalopathy).

The diagnostic method of the invention is particularly important for detecting carriers of CJD, for monitoring treatment of CJD patients and for estimating the patients clinical stage as well as the severity of the disease. It is to be noted that when referring to CJD, all other TSE's are also included. Suspected carriers of pathogenic prion mutations are tested by molecular method for the presence of the mutation, which defines their carrier status. However, and since the age of disease onset can be between 35-85 or more, there is no test to establish at early stages whether the disease is manifesting. Such test could be crucial for early or prophylactic treatment. The detection of carriers of the mutation leading to CJD disease may be used, for example, in genetic counseling.

The method of the invention for detecting and diagnosing human prion disease carriers, preferably CJD carriers, may be modified to detect the presence in urine of specific metabolites of PrP^{SC}, which have now been identified in the urine of CJD patients. Being specific for the human prion diseases, these metabolites can be identified in the urine of the patients, without first subjecting the protein sample obtained from the urine to protease digestion. This modification can employ antibodies specific for PrP^{SC}, as shown in the Examples, or antibodies that are specific for the metabolites that are unique for human prion disease. For example, 6H4 antibodies can bind to a metabolite of PrP which is not present in normal urine. Therefore, the protease digestion could be omitted, and suitable antibodies, for example, the primary antibody 6H4 used, to bind CJD specific metabolites of PrP^{SC}, found only in sick individuals.

Additionally, the diagnostic method of the invention is useful in identifying infection of BSE, particularly in individuals that have been exposed to the disease. Identifying human carriers of BSE has importance, inter alia, in screening blood samples of human donors for the presence of a prion disease in the donors. Screening can be carried out, for example, by obtaining a urine sample from the donor, detecting the presence of the abnormal isoform of prion protein (PrP^{SC}) in the urine sample by the detection method of the invention and ascribing the results of the detection to said blood sample. Such screening would prevent the use of prion-infected blood, thus diminishing risks of blood transfusions.

Additionally, the diagnostic method of the invention, when applied to bovine animals, and also to other domestic animals like sheep and goats or any other animal of interest susceptible to BSE or any other prion disease, may assist in screening food products originating from the tested animals, like meat and dairy products, and reduce the risk of infection of human consumers.

Some steps of the method of the invention may preferably be adapted when applied to bovine animals. First, larger urine volume (about 20-30 ml) should be tested. After dialysis, it is preferred to stabilize the proteins of the dialysate by adding a carrier. Such a carrier may preferably be brain extracts of PrP ablated mice. The dialysate is next precipitated using ultracentrifuge, or preferably methanol precipitation, as indicated above. Following the PK digestion, samples are separated on SDS PAGE, and the blots are blocked using human serum albumin. It is to be appreciated that any other suitable protein precipitation methods such as TCA (Trichloracetic acid), may be used by the method of the invention.

In yet a further embodiment, the invention relates to a diagnostic kit for detecting the presence of the abnormal isoform of prion protein (PrP^{SC}) in a urine sample of a subject, which comprises means for isolating from said urine sample all proteins having a molecular weight higher than about 8 KDa, optionally a carrier for stabilizing the dialysate proteins, a protase for digesting the protein isolate, means for isolating from the digest any non-digested proteins, means for detecting the presence of PrP^{SC} in the non-digested fraction; and instructions for carrying out the detection of the presence of PrP^{SC} in the urine sample.

Specifically, the protease comprised in the kit of the invention may be proteinase K, and the means for detecting the presence of PrP^{SC} may comprise reagents for detecting PrP^{SC} by immunoassay, particularly antibodies that specifically react with the protease-resistant core of PrP^{SC}, such as 3F4 and 6H4.

In another embodiment, the kit of the invention may be modified to exclude the protease, and include antibodies that specifically bind to metabolites of PrP^{SC} that are uniquely found in urine of human prion disease patients and carriers. Preferably, said human prion disease may be CJD or vCJD.

A number of methods of the art of molecular biology are not detailed herein, as they are well known to the person of skill in the art. Such methods include, for example, detection and analysis of naturally occurring, synthetic and recombinant proteins or peptides and the like. Textbooks describing such methods are e.g., Sambrook et al., Molecular Cloning A Laboratory Manual, Cold Spring Harbor Laboratory; ISBN: 0879693096, 1989, Current Protocols in Molecular Biology, by F. M. Ausubel, ISBN: 047150338X, John Wiley & Sons, Inc. 1988, and Short Protocols in Molecular Biology, by F. M. Ausubel et al. (eds.) 3rd ed. John Wiley & Sons; ISBN: 0471137812, 1995. These publications are incorporated herein in their entirety by reference. Furthermore, a number of immunological techniques are not in each instance described herein in detail, as they are well known to the person of skill in the art. See e.g., Current Protocols in Immunology, Coligan et al. (eds.), John Wiley & Sons, Inc., New York, N.Y.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended Claims and equivalents thereof.

Throughout this specification and the Claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It must be noted that, as used in this specification and the appended Claims, the singular forms “a”, “an” and “the” include plural referents unless the content clearly dictates otherwise.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the intended scope of the invention.

### EXAMPLES

### Reagents

### Antibodies

3F4—monoclonal anti PrP antibody, detects the 108-111 amino acids residues in the sequence of the PrP protein [Oesch et al., Cell 40(4):735-736 (1985), Kascsak R J Et al., J. Virol. 61(12):3688-93 (1987)].

6H4—monoclonal anti PrP antibody, which binds to the 144-152 amino acid residues of the PrP protein, purchased from Prionics AG, Switzerland.

### Buffers

Sarkosyl/STE burrer—10 mM Tris HCl pH 7.5, 10 mM NaCl, 1 mM EDTA.

Homogenization buffer—10 mM Tris HCl pH 7.5, 300 mM Sucrose.

TBST buffer—10 mM Tris HCl Ph 8.0, 150 mM NaCl, 0.05% Tween 20.

### Experimental Procedures

### Analysis of Urine Samples

Urine samples (2 ml for hamster; 10 ml for human; 15 ml for bovine) were sedimented for 5 min at 3000 rpm to discard occasional cell debris, and then dialyzed over night in a cellulose tubular membrane (pore range 6000-8000 Dalton, FPI; Texas, U.S.A.) against 5 liters of saline at 4° C. (saline was changed twice during dialysis). For experimental purposes, the dialysis step was omitted in some cases. Subsequently, urine samples were centrifuged at high speed (100000 gₐᵥ *1 hr*4° C.). Pellets were resuspended in 100 μl 2% Sarkosyl/STE buffer. Samples were divided and digested in the presence or absence of proteinase K (PK). Digestion conditions were optimized for each species. For hamster urine: 40 μg/ml PK for 60 min at 37° C.; for human urine: 40 μg/ml PK for 30 min at 37° C.; for bovine urine: 20 μg/ml for 30 min at 37° C.

Following protease digestion, urine samples were boiled in SDS sample buffer.

### Western Blot Analysis

Samples boiled in SDS sample buffer were applied to a 12% SDS PAGE and subsequently transferred to a nitrocellulose membrane. Membranes were blocked with 3% fat milk except for the bovine samples which were blocked with 5% HSA (Human Serum Albumin, Sigma). A second blocking step was performed with a mixture of 1:3000 anti mouse IgG and 1:3000 anti-rabbit 1 gG in TBST buffer (for 30 min) to avoid non specific binding of the secondary Ab to IgG light chain present in some urine samples. Membranes were then rinsed in TBST for 15 min and immunoblotted either with α PrP mAb 3F4 or 6H4 (Hamster, Human) at 1:5000 or 6H4 (Bovine) at 1:5000.

### In Vivo Experiments

Syrian hamsters were inoculated with samples containing urine PrP from normal or scrapie sick hamsters. For inoculation, urine samples were prepared as described above (including PK digestion but not SDS boiling) and diluted as required in 1% BSA/PBS. Brain samples from scrapie infected hamsters were diluted to contain comparable concentrations of PrP^{SC} and inoculated to additional groups of hamsters. To achieve similar concentrations of protease resistant PrP of brain and urine inoculi, each animal was inoculated, depending on the appropriate experimental group, with 50 μl sample containing PrP originating from either 0.5 ml urine or from 1.25 μl of 10% brain homogenate of scrapie hamster.

### Hamster Samples

Following inoculation, animals were examined daily for scrapie associated symptoms. For time course experiments, groups of 3 hamsters in an equivalent stage of disease incubation, were housed each week in a metabolic cage for urine collection from 15:00 p.m. to 08:00 a.m. of the next day. Urine was collected in the morning and immediately was frozen at −80° C. Food and water were supplied ad libitum. Similar procedure was applied to scrapie sick hamsters.

### Human Urine Samples

Most of CJD patients tested (6 out of 8) were genetic patients carrying the E200K mutation [Hsiao, K., et al., N Engl J Med 324(16), 1091-7 (1991); Gabizon, R., et al., Nat Med 2(1), 59-64 (1996); Gabizon, R., et al., Am J Hum Genet 53(4), 828-35 (1993) Goldfarb, L. G., et al., Lancet 336(8715), 637-8 (1990)]. One of the patients was a 52 year old individual homozygous for this mutation [Simon, E. S., et al., Ann Neurol 47(2), 257-60 (2000)]. Among the other genetic patients, 4 were MM at codon 129 and one was MV. The E200K mutation is located at a Methionine 129 allele [Gabizon, R., et al., (1993) ibid]. The human controls (n=15), were either healthy individuals (n=7) or patients suffering from diverse neurological disorders, such as Alzheimer's disease (n=3), multiple sclerosis (n=2) and stroke (n=3).

Whenever possible, human samples from CJD patients and controls were the first morning urine. Some CJD and post stroke patients were bearing catheters, and in these cases urine was collected for a period of up to 8 h in a urine collecting bag. All samples were frozen until further use.

### Bovine Urine Samples

All BSE and most control bovine urine samples were obtained from the Veterinary Laboratory Agency (VLA) in London. The VLA samples constituted 51 samples of 24 cows, all coded for blind testing. Additional freshly frozen control samples were obtained from the Hebrew University Veterinary School. According to VLA records, most samples were frozen following collection while some were kept chilled. No information was provided regarding time of day for sample collection.

### Tissue Homogenates

Whole brain or kidney samples were homogenized in ten volumes homogenization buffer. Following centrifugation [2000 rpm (rounds per minute), 15 min at 4° C.), the supernatant was frozen (−80° C.).

### Example 1

### Immunoblot Analysis of PrP^{SC} in Urine Samples

In order to develop a non-invasive method for diagnosis of different prion diseases, the possibility of detecting the prion protein UPrP^{SC} in urine samples of different mammalian subjects was examined. Urine samples from scrapie infected hamsters, CJD patients, and BSE infected cattle, as well as from their appropriate controls, were processed for enrichment of UPrP^{SC}, and subsequently immunobloted for PrP peptides as described herein above. Human and hamster urine samples were immunoblotted with either mAb 3F4 or 6H4, while bovine samples were blotted only with mAb 6H4. Parallel samples were blotted only with secondary α mouse antisera and showed no interfering signals.

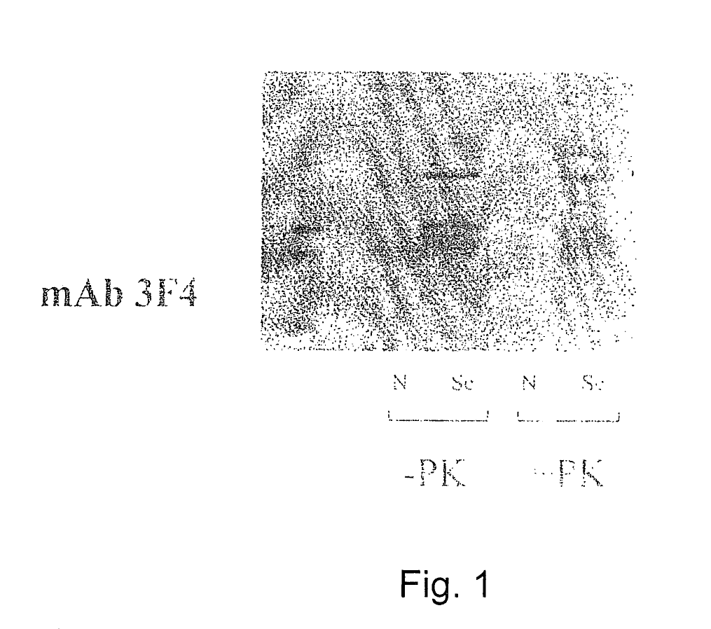

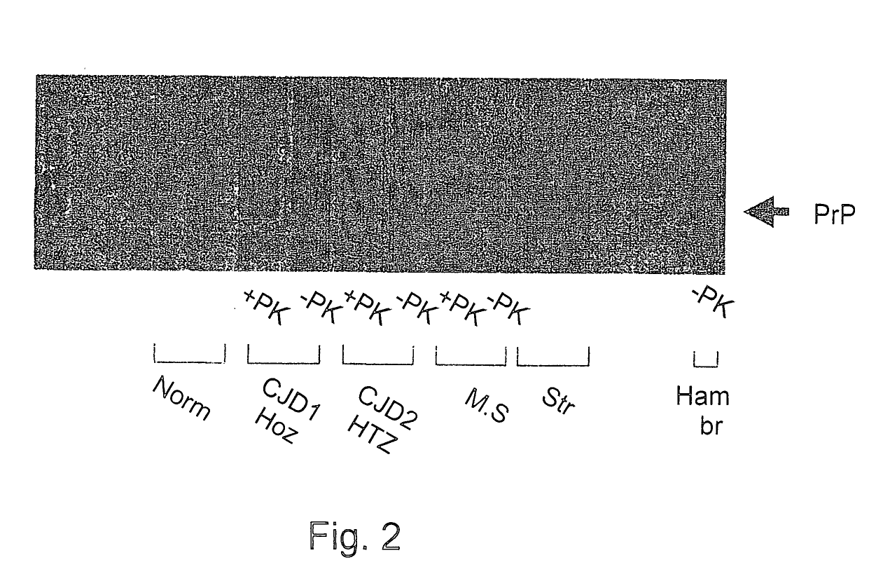

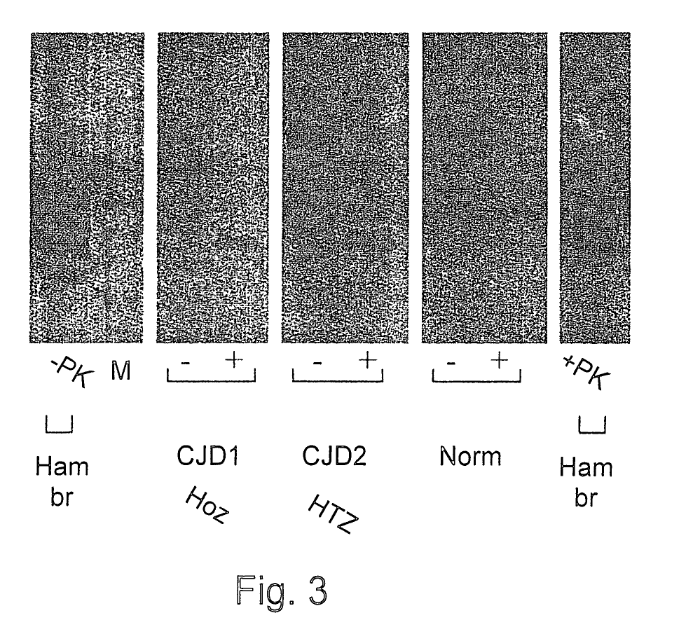

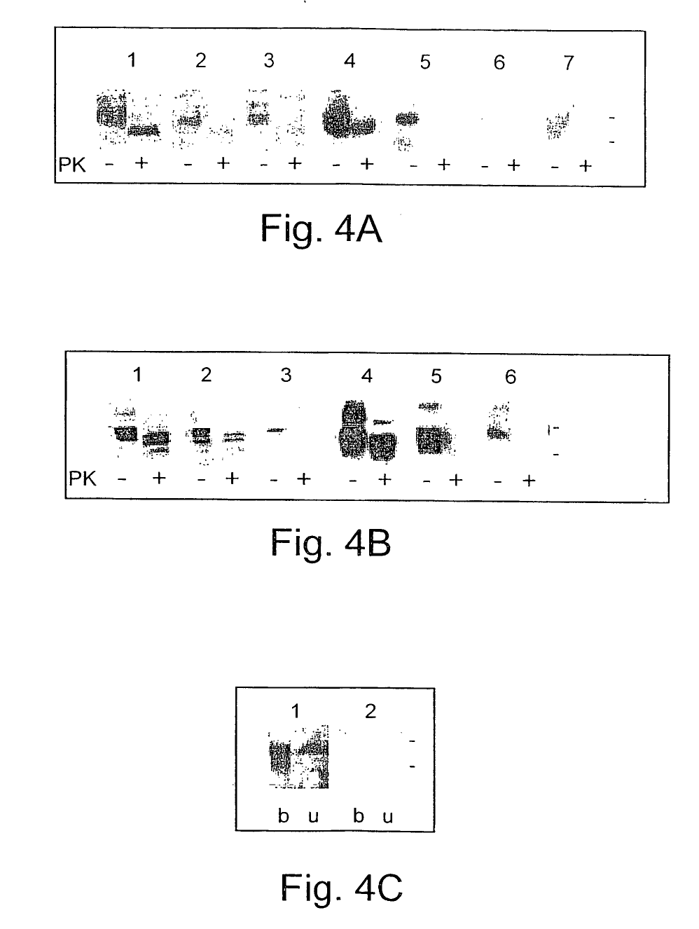

As shown in FIGS. 1, 2, 3 and 4, a precipitable and protease resistant form of PrP could be detected only in the dialyzed urine of prion disease affected humans and animals. However, in urine samples of the appropriate controls, the resistant form of PrP could not be detected. PrP^{SC} was not found in samples from MS (Multiple Sclerosis) patient, Stroke patients as well as in healthy individuals (FIGS. 2 and 3). The differences in band strength of the two patients (FIG. 2) is probably the result of the different clinical status of the patients. Similar results were obtained with other CJD homozygous and heterozygous patients (FIGS. 3 and 4).

In order to verify that the observed signal was specific to PrP, a blocking experiment was next performed. As shown in FIG. 4C, PrP signal in urine could be blocked by the 3F4 peptide, providing strong evidence that this signal belongs to a PrP peptide.

An essential element in developing the method for detecting UPrP^{SC} in urine was concentrating the samples by dialyzing the urine samples prior to their centrifugation and digestion.

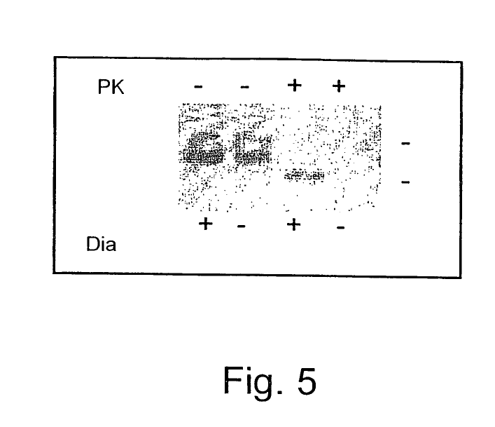

FIG. 5 demonstrates the importance of dialysis of urine samples from scrapie infected hamsters. The protease resistant UPrP^{SC} could be detected after PK digestion only in dialyzed sample.

A surprising result depicted above is that a protease sensitive PrP isoform is present in the precipitable fraction of the normal urine samples, as opposed to what is expected for PrP^{C}. It is to be noted however, that no detergent was added to the urine before ultracentrifugation as performed in membrane extractions that result in a soluble PrP^{C} [Meyer, R. K., et al., Proc Natl Acad Sci U.S.A. 83)8), 2310-4 (1986); Gabizon, R., et al., Proc Natl Acad Sci U.S.A. 84(12), 4017-21 (1987)]. It is also possible that all PrP molecules are present in urine in a partially denatured state due to the presence of variety of denaturing agents such as urea. Also dialysis of normal urine may induce the aggregation of the PrP^{C} isoform which, as opposed to UPrP^{SC}, is protease sensitive. Although the exact chemical nature of UPrP^{SC} is yet to be determined, its molecular weight seems to be slightly higher than full length and fully glycosylated PrP^{C} or PrP^{SC}. In addition, the pattern of UPrP^{SC} in the immunoblots suggest it may be composed mostly of the higher molecular band of PrP, and not of the less glycosylated species. This may indicate that partially or non-glycosylated PrP is less resistant to the conditions encountered by PrP^{SC} before it is excreted in urine as UPrP^{SC}.

It is conceivable that at least for hamsters, UPrP^{SC} did not originate directly from the kidneys, since no PrP^{SC} could be identified in the kidney tissue of scrapie infected hamsters (FIG. 4B, sample 6). This suggests UPrP^{SC} originates from other organs and arrives to the urine from blood.

### Example 2

### Diagnosis of BSE in Urine Samples of Cattle

Twenty-four different samples of cattle urine obtained from England were double blind tested for the presence of PrP^{SC}. Briefly, different samples of 20 ml of cattle urine were processed by dialysis against saline, as described above. The dialyzed samples were further stabilized by adding different concentrations 10-5 μl of a 10% homogenate of brain extracts of PrP ablated mice. Addition of the PrP ablated mice extracts as a carrier, improved the ability to obtain a more concentrated protein precipitate due to the presence of molecules in said extract which bind and stabilize the urine PrP. However, it is to be appreciated that addition of the PrP ablated mice extracts as a carrier is not necessary and the test is feasible also without this additional step. The dialyzed samples were then precipitated with methanol (1:4 volume to volume sample/methanol) and subsequently digested in the presence or absence of PK as described above. Digested samples were then subjected to Western blot analysis (12% SDS PAGE), and blots were blocked using 5% Human Serum Albumin (HSA) in TBST buffer prior to addition of the primary antibody. Table 1 presents the obtained results, compared with clinical diagnosis of the same samples by brain histopatholgy. As demonstrated in Table 1, the results obtained by this experiment were highly significant. All the negative samples were properly diagnosed and most of the clinically affected animals were diagnosed as BSE positives (10 out of 12) by the method of the present invention. Only four samples (Nos. 4, 9, 14 and 22) were inconclusive, probably due to non-optimized storage and shipment conditions. Further optimization of the sample storage and handling conditions is within the scope of the present invention.

TABLE 1
BSE diagnosis of cattle urine samples

| | | Urine Test according to | |
|---|---|---|---|
| Sample # | | the Invention | Brain Histopathology |
| 1 | a | + | + |
| | b | + | |
| 2 | a | − | − |
| | b | − | |
| 3 | a | − | − |
| | b | − | |
| 4 | a | +/− | |
| | b | +/− | − |
| | c | + | |
| 5 | a | − | − |
| | b | − | |
| 6 | a | − | − |
| | b | − | |
| 7 | a | + | + |
| | b | + | |
| 8 | a | − | − |
| | b | − | |
| 9 | a | +/− | |
| | b | + | − |
| | c | +/− | |
| 10 | a | + | + |
| | b | + | |
| 11 | a | + | + |
| | b | + | |
| 12 | a | − | |
| | b | − | − |
| | c | − | |
| 13 | a | + | + |
| | b | + | |
| 14 | a | +/− | + |
| | b | +/− | |
| 15 | a | − | |
| | b | − | − |
| | c | − | |
| 16 | a | − | − |
| | b | − | |
| 17 | a | + | + |
| | b | + | |
| 18 | a | +/− | + |
| | b | +/− | |
| 19 | a | + | + |
| | b | + | |
| 20 | a | + | + |
| | b | + | |
| 21 | a | + | + |
| | b | + | |
| 22 | a | Ns | + |
| | b | Ns | |
| 23 | | − | − |
| 24 | a | − | − |
| | b | − | |

(+) positive
(−) negative
(+/−) suspected (low signal)

Ns—non specific background

### Example 3

### Comparison Between 3F4 and 6H4 Antibodies in the Analysis of PrP^{SC} in Urine Samples

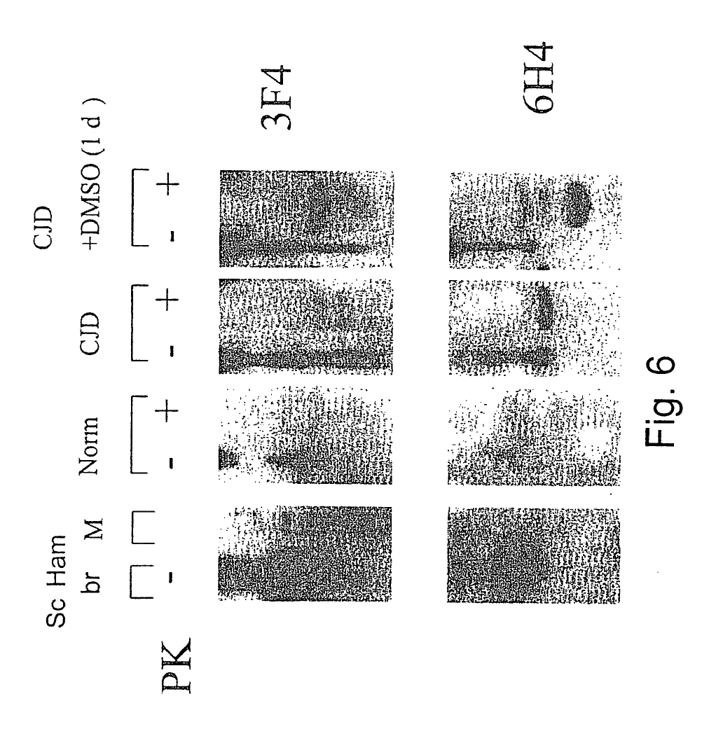

3F4 and 6H4 monoclonal antibodies were used to detect PrP^{SC} in urine samples of CJD patients. To precipitate the samples, methanol was used instead of ultracentrifugation. As shown in FIG. 6, 6H4 antibodies could detect two additional lower bands, probably representing two additional metabolites of PrP that are PK-resistant and are present only in CJD patients. The additional metabolites detected by the 6H4 antibodies were found only in CJD patients, even when the treatment with PK was omitted [FIG. 6].

A considerable increase in the amount of PrP secreted in the urine was found when dimethylsulfoxide (DMSO) was administrated to CJD patients prior to examination. FIG. 6 shows the result of Western blot analysis of CJD patient with and without DMSO administration (5 ml, three times daily) for one day. As depicted in the right panel of FIG. 6, DMSO led to the enhancement of PrP secretion to the urine.

### Example 4

### Detection of the Protease Resistant UPrP^{SC} in a Urine Sample Prior to Onset of Clinical Symptoms

Detection of PrP^{SC} at the final stages of prion disease may result from some degree of blood brain barrier disruption by brain degeneration [De Armond, S. J., et al., Prog Clin Biol Res 317, 601-18 (1989)]. However, the presence of PrP^{SC} in prion infected urine in early stage of the incubation time, suggests a clearance pathway for the aberrant PrP protein either from brain or from peripheral organ, through its excretion into urine. To address this question, Syrian hamsters were inoculated either intra-cerebrally (i.c.) or intraperitoneally (i.p.) with hamster prions. Urine samples were collected every week during the incubation period, as described in experimental procedures and each sample was frozen immediately after collection. At the end of the experiment, similar volumes of these urine samples were thawed, enriched for PK resistant UPrP^{SC} as described above and subsequently immunoblotted with the anti PrP mAb 3F4.

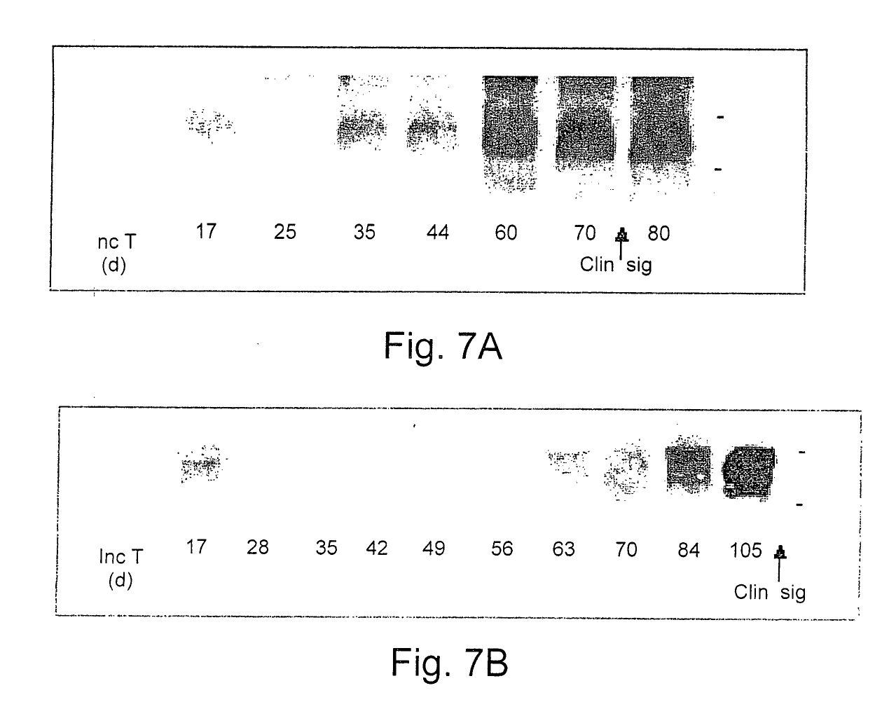

As can be seen in FIG. 7, a light signal of prion specific protease resistant PrP was detected in urine samples of i.c. inoculated hamsters after only 17 days (FIG. 7A), following by the disappearance of the PrP signal until day 35. Subsequently, this signal increased from day 35 until the appearance of clinical signs. Similar results were obtained for i.p. inoculated hamsters (FIG. 7B). A PrP signal was detected in the first weeks following inoculation, disappeared at later dates and reappeared at about 60 days. These results may infer that some of the prion inoculum is immediately secreted following inoculation. Thereafter, until the first stages of prion protein accumulation in brain, no PrP signal appeared in urine.

It is to be noted that the reported incubation time for i.c. or i.p. scrapie inoculated hamsters with the 263 strain, is about 75 and 120 days respectively, and PrP^{Sc} can be identified in enriched brain samples of these hamsters at about 40 (i.c.) or 70 (i.p.) days [Czub, M., Braig, H. R., and Diringer, H. J Gen Virol 69 (Pt 7), 1753-6 (1988); Czub, M., Braig, H. R., and Diringer, H. J Gen Virol 67 (Pt 9), 2005-9 (1986); Taraboulos, A., et al., Proc Natl Acad Sci U.S.A. 89, 7620-7624 (1992)].

These results demonstrate that UPrP^{SC} is excreted in urine parallel to its accumulation in brain.

These results clearly indicate that urine testing for protease resistant PrP can be used to diagnose prion diseases in animals and humans at terminal stages of the disease and also can be used to diagnose prion diseases in subclinical stages of infection. Detection of the PrP signal at the first weeks post infection is due to clearance of the inoculum and therefore, the PrP urine test may serve as a powerful tool to diagnose a potential new occurrence of infection. This may provide in future, an effective anti-prion therapy for the treatment of individuals at risk of a new prion exposure.

### Example 5

### Can Prion Disease Be Transmitted by UPrP^{SC}

Detection of UPrP^{SC} in urine during early stages of incubation time and before the appearance of clinical signs, as was showed herein above, raises the alarming possibility that transmission of prion diseases may occur via urine of either ill animals or of animals during the incubation time of the prion diseases. This prospect is especially disturbing in the case of BSE infected cattle as well as in natural scrapie in sheep, since the mechanism by which these diseases are transmitted among animals within the herd was never elucidated [Chatelain, J., and Dautheville Guibal, C. Eur J Epidemiol 5(1), 113-6 (1989); Berne, R. M., and Levy, M. N. Physiology, 4th Ed. (1998)]. Thus, it is conceivable that urine may contaminate the dwelling areas of these animals.

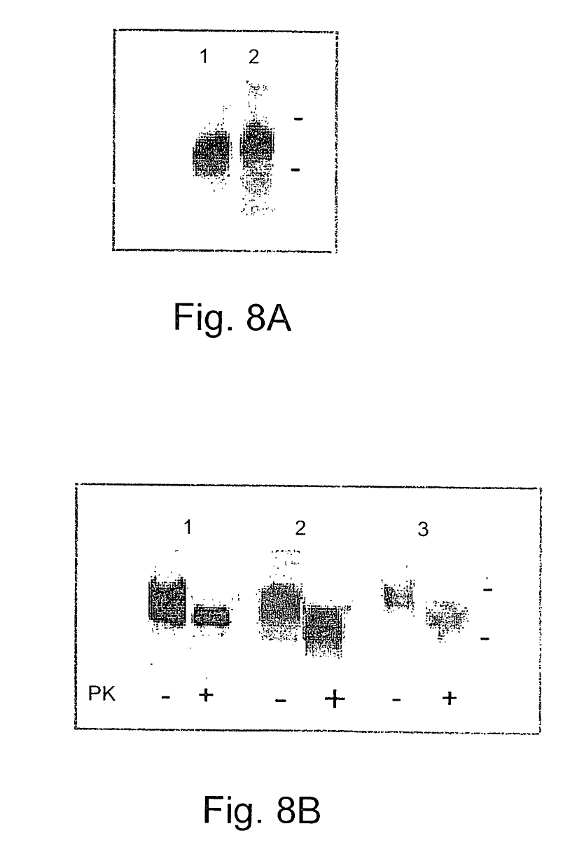

To investigate whether urine from TSE infected animals can be infectious, twenty hamsters were inoculated with UPrP^{SC} pooled and enriched from urine of 10 hamsters terminally will with scrapie. Twenty hamsters were inoculated with similarly prepared samples from 10 normal hamsters as negative control. Brain samples from scrapie infected hamsters, diluted to PrP^{SC} concentrations (1.25 μl of 10% homogenate) comparable to those of the enriched UPrP^{SC} (from 0.5 ml urine), were inoculated to additional groups of hamsters (FIG. 8A) as a positive control. Hamsters were observed daily for symptoms of scrapie infection and urine was collected periodically from animals inoculated with UPrP^{SC}. Some of the hamsters inoculated with UPrP^{SC} were sacrificed, at different time points during the experiment and tested for the presence of PrP^{SC} in their brains.

As expected, animals inoculated with scrapie infected brain samples suffered from fatal disease symptoms at about 80 days post inoculation (dpi). Contrarily, none of the animals inoculated with urine samples (normal or scrapie infected) developed clinical symptoms of prion disease to date (270 dpi). Twelve hamsters (4 groups of 3) were tested for the presence of UPrP^{SC} and all were found positive from about 60days post inoculation (FIG. 8B, lane 2). In addition, low concentrations of PrP^{SC} could be identified in brain of one out of three urine infected hamsters that were sacrificed at about 120 days (FIG. 8B, lane 3). All other hamsters in this experiment are still under observation to determine whether they will develop a fatal prion disease at a later date. These results suggest that UPrP^{SC} inoculation can result in a subclinical or carrier state prion infection.

While specific embodiments of the invention have been described for the purpose of illustration, it will be understood that the invention may be carried out in practice by skilled persons with many modifications, variations and adaptations, without departing from its spirit or exceeding the scope of the Claims.

1-46. (canceled)

47. A method for detecting the presence of the abnormal isoform of prion protein (PrP^{SC}) in a urine sample of a subject, said method comprising the steps of:
a. providing a urine sample of said subject;
b. isolating from said sample proteins; and
c. detecting the presence of PrP^{SC} in the protein mixture obtained in step (b) by a suitable detection technique.

48. A method according to claim 47 further comprising the step of subjecting the proteins obtained in step (b) to protease digestion.

49. A method according to claim 48 for detecting the presence of the abnormal isoform of prion protein (PrP^{SC}) in a urine sample of a subject, said method comprising the steps of:
a. providing a urine sample of said subject;
b. isolating from said sample all proteins having a molecular weight higher than 8 KDa;
c. subjecting the proteins obtained in step (b) to protease digestion;
d. isolating from the mixture obtained in step(c) any protease resistant proteins; and
e. detecting the presence of PrP^{SC} in the protease resistant fraction obtained in step (d) by a suitable detection technique.

50. A method according to claim 49 wherein in step (b) said proteins are isolated by subjecting the urine sample to dialysis and precipitating the proteins from the dialysate.

51. A method according to claim 50 wherein step (b) further comprises addition of a carrier to the dialysate, prior to the protein precipitation.

52. A method for detecting the presence of the abnormal isoform of prion protein (PrP^{SC}) in a urine sample of a subject, said method comprising the steps of:
a. providing a urine sample of said subject;
b. isolating from said sample all proteins having a molecular weight higher than about 8 KDa by subjecting said sample to dialysis, wherein said dialysis is performed using a membrane having a pore range of from about 6 KDa to 8 KDa;
c. precipitating said proteins by ultracentrifuging the dialysate;
d. subjecting the proteins obtained in step (b) to protease digestion;
e. isolating from the mixture obtained in step (c) any protease resistant proteins; and
f. detecting the presence of PrP^{SC} in the protease resistant fraction obtained in step (d) by a suitable detection technique.

53. A method for detecting the presence of the abnormal isoform of prion protein (PrP^{SC}) in a urine sample of a subject, said method comprising the steps of:
a. providing a urine sample of said subject;
b. isolating from said sample all proteins having a ;molecular weight higher than about 8 KDa by subjecting said sample to dialysis, wherein said dialysis is performed using a membrane having a pore range of from about 6 KDa to 8 DKa;
c. precipitating said proteins by ultracentrifuging the dialysate for 1 hour at 100,000×g at 4° C.;
d. subjecting the proteins obtained in step (b) to protease digestion;
e. isolating from the mixture obtained in step (c) any protease resistant proteins; and
f. detecting the presence of PrP^{SC} in the protease resistant fraction obtained in step (d) by a suitable detection technique.

54. A method according to claim 52 wherein the proteins are precipitated by any one of methanol and TCA.

55. A method according to claim 54 wherein the proteins are precipitated by methanol.

56. A method according to claim 52 wherein said protease is proteinase K.

57. A method according to claim 53 wherein said protease is proteinase K.

58. A method according to claim 56 wherein in step (e) the presence of the PrP^{SC} protease-resistant core in said protease resistant fraction, is detected by immunoassay.

59. A method according to claim 58 wherein said immunoassay is by immunoblot SDS PAGE analysis.

60. A method according to claim 60 wherein said PrP antibodies are 3F4 or 6H4 monoclonal antibodies.

61. A method according to claim 60 wherein said PrP antibodies are 3F4 or 6H4 monoclonal antibodies.

62. A method for diagnosing a prion disease in a subject comprising the steps of:
a. obtaining a urine sample of said subject; and
b. detecting the presence of the abnormal isoform of prion protein (PrP^{SC}) in said urine sample by the method of claim 52;
whereby the presence the PrP^{SC} protein in said sample indicates that said subject carries a prion disease.

63. A method for diagnosing a prion disease in a subject comprising the steps of:
a. obtaining a urine sample of said subject; and
b. detecting the presence of the abnormal isoform of prion protein (PrP^{SC}) is said urine sample by the method of claim 53;
whereby the presence the PrP^{SC} protein in said sample indicates that said subject carries a prion disease.

64. A method of claim 62 wherein said prion disease is a TSE disease.

65. A method claim 64 wherein said subject is a human subject.

66. A method of claim 64 wherein said subject is a bovine animal.

67. A method of claim 66 wherein said prion disease is BSE.

68. A method according to claim 62 wherein diagnosing of said prion disease is prior to or after onset of clinical symptoms.

69. A method for detecting the presence of metabolites of the abnormal isoform of prion protein (PrP^{SC}) in a urine sample of a human subject, said metabolites being unique for human prion disease carries, said method comprising the steps of:
a. providing a urine sample of said subject;
b. isolating from said sample all proteins having a molecular weight higher than 8 KDa; and
c. detecting the presence of said metabolites of PrP^{SC} in the protein sample obtained in step (b) by an immunoassay comprising the use of 6H4 monoclonal antibodies that specifically bind to the protease-resistant core of PrP^{SC} found in the urine of human prion disease carriers.

70. A method according to claim 69 wherein step (b) said proteins are isolated by subjecting the urine sample to dialysis using a membrane having a pore range of from 6 Kda to 8 Kda, and precipitating the proteins from the dialysate by ultracentrifugation.

71. A method according to claim 70 wherein the precipitation is performed by ultracentrifuging the dialysate for about 1 hour at 100,000×g at 4° C.

72. A method according to claim 69 wherein the proteins are precipitated by any one of methanol and TCA.

73. A method according to claim 72 wherein the proteins are precipitated by methanol.

74. A method according to claim 69 wherein said human prion disease is CJD.

75. A method for diagnosing a human prion disease in a subject comprising the steps of:
a. obtaining a urine sample of said subject; and
b. detecting the presence of metabolites of the abnormal isoform of prion protein (PrP^{SC}) that are unique for human prion disease patients in said urine sample by the method of claim 69;
whereby the presence of said PrP^{SC} protein metabolites in said sample indicates that said subject carries a human prion disease.

76. A method according to claim 75 wherein said human prion disease is CJD.

77. A diagnostic kit for detecting the presence of the abnormal isoform of prion protein (PrP^{SC}) in a urine sample of a subject, said kit comprising:
a. means for isolating from said urine sample proteins;
b. optionally, suitable carrier for stabilizing the PrP^{SC} in the urine sample.
c. a protease for digesting the protein isolate obtained by (a) or (b);
d. means for isolating from the digest by (c) any protease resistant proteins;
e. means for detecting the presence of PrP^{SC} in the protease resistant fraction obtained by (d) and
f. instructions for carrying out the detection of the presence of PrP^{Sc} in the urine sample according to the method of claim 52.

78. A kit according to claim 77 wherein said means for isolating proteins is for isolating proteins having a molecular weight higher than 8 Kda.

79. A kit according to claim 78 wherein said protease is proteinase K.

80. A kit according to claim 77 wherein said means for detecting the presence of PrP^{SC} comprise reagents for detecting PrP^{SC} by immunoassay.

81. A kit according to claim 80 wherein said immunoassay reagents comprise antibodies that specifically react with the protease-resistant core of PrP^{SC}.

82. A diagnostic kit for detecting the presence of metabolites of the abnormal isoform or prion protein (PrP^{SC}) that are unique for human prion disease carriers in a urine sample of a human subject, said kit comprising:
a. means for isolating from said urine sample all proteins having a molecular weight higher than about 8 KDa;
b. means for detecting the presence of PrP^{SC} metabolites that are unique for human prion disease carriers in the protein sample obtained by step (a) by immunoassay comprising antibodies that specifically react with the metabolites of PrP^{SC} that are unique for human prion disease carriers; and
c. instructions for carrying out the detection of the presence of PrP^{SC} in the urine sample according to the method of claim 69.

83. A kit according to claim 82 wherein said human prion disease is CJD.
